SEQUENCE LISTING (11 sequences)

SEQ ID NO: 1 - protein, 4 aa, Artificial
  chemically synthesized
REDV

SEQ ID NO: 2 - protein, 5 aa, Artificial
  chemically synthesized
YIGSR

SEQ ID NO: 3 - protein, 7 aa, Artificial
  chemically synthesized
RYVVLPR

SEQ ID NO: 4 - protein, 12 aa, Artificial
  chemically synthesized
TAGSCLRKFSTM

SEQ ID NO: 5 - protein, 8 aa, Artificial
  chemically synthesized
WQPPRARI

SEQ ID NO: 6 - protein, 6 aa, Artificial
  chemically synthesized
PRRARV

SEQ ID NO: 7 - protein, 6 aa, Artificial
  chemically synthesized
PRRGRV

SEQ ID NO: 8 - protein, 19 aa, Artificial
  chemically synthesized
YEKPGSPPREVVPRPRPGV

SEQ ID NO: 9 - protein, 28 aa, Artificial
  chemically synthesized
RPSLAKKQRFRHRNRKGYRSQRGHSRGR

SEQ ID NO: 10 - protein, 17 aa, Artificial
  chemically synthesized
RIQNLLKITNLRIKFVK

SEQ ID NO: 11 - protein, 20 aa, Artificial
  chemically synthesized
RYVVLPRPVDFEKGMNYTVR

(19) United States
(12) Patent Application Publication
(10) Pub. No.: US 2009/0324685 A1
(43) Pub. Date: Dec. 31, 2009

(54) MEDICAL DEVICE COATINGS CONTAINING CHARGED MATERIALS

(75) Inventors: WAYNE FALK, ALAMEDA, CA (US); MICHELE ZOROMSKI, MINNEAPOLIS, MN (US); ROBERT W. WARNER, WOODBURY, MN (US); LILIANA ATANASOSKA, EDINA, MN (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., MAPLE GROVE, MN (US)

(21) Appl. No.: 12/491,434

(22) Filed: Jun. 25, 2009

Related U.S. Application Data
(60) Provisional application No. 61/075,777, filed on Jun. 26, 2008.

(51) Int. Cl. A61F 2/04; A61K 38/06

(52) U.S. Cl. 424/426; 424/423; 514/18

(57) ABSTRACT

In accordance with certain aspects of the present invention, medical devices are provided which are configured for implantation or insertion into a subject. The medical devices include at least one coating region that comprises (a) a charged polyamino-acid-containing polymer having a first net charge and (b) an additional charged polymer having a second net charge that is opposite in sign to that of the first net charge. The additional charged polymer may or may not be a polyamino-acid-containing polymer.

### RELATED APPLICATIONS

This application claims priority from U.S. provisional application 61/075,777, filed Jun. 26, 2008, which is incorporated by reference herein in its entirety.

### FIELD OF THE INVENTION

The present invention relates to coatings for implantable and insertable medical devices.

### BACKGROUND

Medical devices may be implanted or inserted into the body of a patient to provide any of a number of functions in the body including, for example, mechanical support, therapeutic agent delivery, tissue scaffolding and/or electrical stimulation, among other functions.

In some instances, it is desirable to promote healthy tissue growth on a given medical device surface, for example, in order to render the device more biocompatible. As a specific example, for medical devices that are implanted or inserted into the vasculature, it may be desirable to provide a device surface that promotes the formation of a functional endothelial cell layer. A functional endothelial cell layer is known to be effective in reducing or eliminating inflammation and thrombosis, which can occur in conjunction with the implantation of foreign bodies in the vasculature. See, e.g., J. M. Caves et al., J. Vasc. Surg. (2006) 44: 1363-8.

Cells in their natural environment are anchored by discrete attachments to adhesion proteins in the extracellular matrix. The primary interaction between cells and adhesion proteins is believed to occur via integrins (heterodimeric receptors in the cell membrane), and integrin binding domains of the adhesion proteins. The molecular recognition of cells by synthetic materials can be achieved by integrating the functional sequence contained in the adhesion proteins. J. A. Hubbell, “Materials as morphogenetic guides in tissue engineering,” Current Opinion in Biotechnology 14 (2003) 551-558. This sequence can be as short as three amino acids, such as in the well-studied RGD sequence which is known to bind fibronectin. Many other integrin and non-integrin peptide binding sequences have been discovered and are currently being developed for use in biomaterials. See, e.g., E. Genove et al., Biomaterials 26 (2005) 3341-3351 and the references cited therein. Animal models have shown beneficial effects of these materials. R. Blindt et al., J. Am. Coll. Cardiol. 47 (2006) 1786-95 N. J. Turner et al., Circulation. 114 (2006) 820-829; and B. P. Chan et al., Journal of Biomedical Materials Research Part B: Applied Biomaterials, 72B (1) (2004) 52-63.

In vitro cell culture and cell adhesion assays on NO releasing biomaterials have shown increased endothelial cell (EC) proliferation, smooth muscle cell (SMC) inhibition and a reduction in platelet and inflammatory cell adhesion, suggesting improved endothelialization, reduced neointimal growth and improved thromboresistance in vivo. See K. S. Bohl Masters et al., J. Biomater. Sci. Polymer Edn, 16 (5), 2005, 659-672, M. C. Frost et al., Biomaterials 26 (2005) 1685-1693, and Ho-Wook Jun et al., Biomacromolecules, 6 (2005) 838-844. In several animal models, placement of NO releasing materials at the site of vascular injury has been shown to virtually eliminate the incidence of intimal hyperplasia. See Bohl Masters et al. and Jun et al., supra, as well as the references cited therein.

### SUMMARY OF THE INVENTION

In accordance with certain aspects of the present invention, medical devices are provided which are configured for implantation or insertion into a subject. The medical devices include at least one coating region that comprises (a) a charged polyamino-acid-containing polymer having a first net charge and (b) an additional charged polymer having a second net charge that is opposite in sign to that of the first net charge. The additional charged polymer may or may not be a polyamino-acid-containing polymer.

According to certain other aspects of the present invention, methods are provided for making such medical devices. These methods include methods that comprise applying a series of charged materials over a substrate surface, wherein each successive charged material in the series has a net charge that is opposite in sign to the net charge of the previously applied material.

Advantages of the present invention include one or more of the following, among others: (a) enhanced endothelial cell attachment and growth, (b) smooth muscle cell inhibition, (c) coating biodegradability, (d) excellent control of coating thickness and uniformity, and (e) tailored immobilization of bioactive molecules/functional groups at selected (i.e., site specific) coating regions.

These and other aspects, embodiments and potential advantages of the present invention will become immediately apparent to those of ordinary skill in the art upon reading the Detailed Description to follow.

### BRIEF DESCRIPTION OF THE FIGURES

FIG. 1A is a schematic view of a stent in accordance with an embodiment of the present invention.

FIG. 1B is schematic view of a cross section taken along line b-b of FIG. 1A.

### DETAILED DESCRIPTION

In accordance with certain aspects of the present invention, medical devices are provided which are configured for implantation or insertion into a subject. The medical devices include at least one coating region that comprises (a) a charged polyamino-acid-containing polymer having a first net charge and (b) an additional charged polymer having a second net charge that is opposite in sign to that of the first net charge, which additional charged polymer may or may not be a polyamino-acid-containing polymer.

In certain embodiments, the coating region comprises a charged polymer that promotes cellular coverage of the medical device surface (e.g., by promoting cell binding and/or cell proliferation, among other possible effects), for instance, a charged polymer that comprises one or more peptide sequences that promote cellular coverage. In certain embodiments, the coating region comprises a charged polymer that releases nitric oxide (NO), which charged polymer may or may not be a charged polyamino-acid-containing polymer. In certain embodiments, the coating region comprises a charged polyamino-acid-containing polymer that releases NO and that comprises one or more peptide sequences that promote cellular coverage.

The coating regions of the invention may be provided over all or only a portion of a substrate surface. The coating regions may be provided in any shape or pattern (e.g., in the form of a series of rectangles, stripes, or any other continuous or non-continuous pattern). Techniques by which patterned coating regions may be provided are described below and include ink jet techniques, stamping techniques roll coating techniques, masking-based techniques, and so forth. Hence, multiple coating regions may be provided at different locations over the substrate surface. These regions may be the same as one another, or they may differ from one another.

As used herein, “polymers” are molecules containing multiple copies (e.g., 5 to 10 to 25 to 50 to 100 to 250 to 500 to 1000 or more copies) of one or more constitutional units, commonly referred to as monomers. As used herein, the term “monomers” may refer to free monomers and to those that have been incorporated into polymers, with the distinction being clear from the context in which the term is used.

Polymers may take on a number of configurations, which may be selected, for example, from cyclic, linear and branched configurations. Branched configurations include star-shaped configurations (e.g., configurations in which three or more chains emanate from a single branch point), comb configurations (e.g., configurations having a main chain and a plurality of side chains), dendritic configurations (e.g., arborescent and hyperbranched polymers), and so forth.

As used herein, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating) and block copolymers. As used herein, a “polymer block” is a portion of a polymer. Polymer blocks include homopolymer blocks and copolymer blocks.

Examples of implantable or insertable medical devices upon which coating regions in accordance with the present invention may be formed include, for example, stents (including coronary vascular stents, peripheral vascular stents such as cerebral stents, urethral, ureteral, biliary, tracheal, gastrointestinal and esophageal stents), stent grafts, vascular grafts, abdominal aortic aneurysm (AAA) devices (e.g., AAA stents, AAA grafts, etc.), vascular access ports, dialysis ports, embolization devices including cerebral aneurysm filler coils (including Guglilmi detachable coils and metal coils), myocardial plugs, septal defect closure devices, patches, catheters (e.g., renal or vascular catheters including balloon catheters), guide wires, balloons, filters (e.g., vena cava filters and mesh filters for distil protection devices), pacemakers, lead coatings including coatings for pacemaker leads, defibrillation leads and coils, ventricular assist devices including left ventricular assist hearts and pumps, total artificial hearts, shunts, valves including heart valves and vascular valves, anastomosis clips and rings, cochlear implants, tissue bulking devices, tissue engineering scaffolds for cartilage, bone, skin and other in vivo tissue regeneration, tissue staples and ligating clips at surgical sites, cannulae, metal wire ligatures, urethral slings, hernia “meshes”, artificial ligaments, joint prostheses, orthopedic prosthesis such as bone grafts, bone plates, fins and fusion devices, orthopedic fixation devices such as interference screws in the ankle, knee, and hand areas, tacks for ligament attachment and meniscal repair, rods and pins for fracture fixation, screws and plates for craniomaxillofacial repair, and dental devices such as dental implants, as well as various other substrates (which can comprise, for example, glass, metal, polymer, ceramic and combinations thereof) which have coatings in accordance with the invention and which are implanted or inserted into the body.

The medical devices of the present invention include medical devices that are used for diagnostics, for systemic treatment, or for the localized treatment of any mammalian tissue or organ. Examples include tumors; organs including the heart, coronary and peripheral vascular system (referred to overall as “the vasculature”), lungs, trachea, esophagus, brain, liver, kidney, bladder, urethra and ureters, eye, nervous system, intestines, stomach, pancreas, ovary, and prostate; skeletal muscle; smooth muscle; breast; dermal tissue; cartilage; and bone.

As used herein, “treatment” refers to the prevention of a disease or condition, the reduction or elimination of symptoms associated with a disease or condition, or the substantial or complete elimination a disease or condition. Typical subjects are vertebrate subjects, more typically mammalian subjects including human subjects, pets and livestock.

It is known that coatings can be formed on substrates based on electrostatic self-assembly of charged materials. In these processes, for example, a first charged material having a first net charge is typically deposited from a first solution onto an underlying charged substrate, followed by deposition of a second charged material (which has a second net charge that is opposite in sign to the net charge of the first material) from a second solution, and so forth. The net charge on the outer layer is reversed upon deposition of each sequential layer. Commonly, 5 to 10 to 25 to 50 to 100 to 200 or more layers may be applied in this technique, depending on the desired thickness of the coating. Examples of charged materials include charged large molecules (e.g., charged polymers), charged small molecules (e.g., charged non-polymeric therapeutic agents), and charged particles, among others. For further information concerning layer-by-layer electrostatic self-assembly methods, see, e.g., US 2005/0208100 to Weber et al., and WO/2005/115496 to Chen et al.

Certain substrates are inherently charged and thus readily lend themselves to electrostatic layer-by-layer assembly techniques. To the extent that the substrate does not have an inherent net surface charge, a surface charge may nonetheless be provided. For example, where the substrate to be coated is conductive (e.g., a metallic substrate), a surface charge may be provided by applying an electrical potential to the same. As another example, a substrate can be provided with a charge by covalently coupling to species having functional groups with a positive net charge (e.g., amine, imine or other basic/cationic groups) or a negative net charge (e.g., carboxylic, phosphonic, phosphoric, sulfuric, sulfonic, or other acidic/anionic groups). Further information on covalent coupling may be found, for example, in Pub. No. US 2005/0002865. In many embodiments, a surface charge may be provided on a substrate simply by adsorbing charged species to the surface of the substrate as a first charged layer. Polyethyleneimine (PEI) is commonly used for this purpose, as it strongly promotes adhesion to a variety of substrates. Further information can be found in Pub. No. US 2007/0154513 to Atanasoska et al.

Regardless of the method by which a given substrate is provided with a surface charge, once a sufficient net surface charge is provided (e.g., via application of an electrical potential, chemical conversion of the surface, adsorption/binding of charged species onto the surface, etc.), the substrate can be readily coated with materials of alternating net charge. In the present invention, those charged materials include charged polymers.

“Charged polymers” are polymers having multiple charged groups. (Such polymers may also be referred to herein as “polyelectrolytes.”) Charged polymers thus include a wide range of species, including polycations and their precursors (e.g., polybases, polysalts, etc.), polyanions and their precursors (e.g., polyacids, polysalts, etc.), ionomers (charged polymers in which a small but significant proportion of the constitutional units carry charges), and so forth. Typically, the number of charged groups is so large that the polymers are soluble in polar solvents (particularly water) when in ionically dissociated form (also called polyions). Some charged polymers have both anionic and cationic groups (e.g., peptides, proteins, etc.) and may have a negative net charge (e.g., because the anionic groups contribute more charge than the cationic groups), a positive net charge (e.g., because the cationic groups contribute more charge than the anionic groups), or may have a neutral net charge (e.g., because the cationic groups and anionic groups contribute equal charge). In this regard, the net charge of a particular charged polymer may change with the pH of its surrounding environment. Charged polymers containing both cationic and anionic groups may be categorized herein as either polycations or polyanions, depending on which groups predominate. (Clearly, charged polymers with only anionic groups have a negative net charge, while charged polymers having only cationic groups have a positively net charge).

Specific examples of polycations include, for instance, polyamines, including poly(amino methacrylates) including poly(dialkylaminoalkyl methacrylates) such as poly(dimethylaminoethyl methacrylate) and poly(diethylaminoethyl methacrylate), polyvinylamines, polyvinylpyridines including quaternary polyvinylpyridines such as poly(N-ethyl-4-vinylpyridine), poly(vinylbenzyltrimethylamines), polyallylamines such as poly(allylamine hydrochloride) (PAH) and poly(diallyldialklylamines) such as poly(diallyldimethylammonium chloride), polyamidoamines, polyimines including polyalkyleneimines such as polyethyleneimines, polypropyleneimines and ethoxylated polyethyleneimines, polycationic peptides and proteins, including histone peptides and homopolymer and copolymers containing basic amino acids such as lysine, arginine, ornithine and combinations thereof, gelatin, albumin, protamine and protamine sulfate, spermine, spermidine, hexadimethrene bromide (polybrene), and polycationic polysaccharides such as cationic starch and chitosan, as well as copolymers, salts, derivatives and combinations of the preceding, among various others.

Specific examples of polyanions include, for instance, polysulfonates such as polyvinylsulfonates, poly(styrenesulfonates) such as poly(sodium styrenesulfonate) (PSS), sulfonated poly(tetrafluoroethylene), sulfonated polymers such as those described in U.S. Pat. No. 5,840,387, including sulfonated styrene-ethylene/butylene-styrene triblock copolymers, sulfonated styrenic homopolymers and copolymers such as a sulfonated versions of the polystyrene-polyolefin copolymers described in U.S. Pat. No. 6,545,097 to Pinchuk et al., which polymers may be sulfonated, for example, using the processes described in U.S. Pat. No. 5,840,387 and U.S. Pat. No. 5,468,574, as well as sulfonated versions of various other homopolymers and copolymers, polysulfates such as polyvinylsulfates, sulfated and non-sulfated glycosaminoglycans as well as certain proteoglycans, for example, heparin, heparin sulfate, chondroitin sulfate, keratan sulfate, dermatan sulfate, polycarboxylates such as acrylic acid polymers and salts thereof (e.g., ammonium, potassium, sodium, etc.), for instance, those available from Atofina and Polysciences Inc., methacrylic acid polymers and salts thereof (e.g., EUDRAGIT, a methacrylic acid and ethyl acrylate copolymer), carboxymethylcellulose, carboxymethylamylose and carboxylic acid derivatives of various other polymers, polyanionic peptides and proteins such as homopolymers and copolymers of acidic amino acids such as glutamic acid, aspartic acid or combinations thereof, homopolymers and copolymers of uronic acids such as mannuronic acid, galatcuronic acid and guluronic acid, and their salts, alginic acid and its salts, hyaluronic acid and its salts, gelatin, carrageenan, polyphosphates such as phosphoric acid derivatives of various polymers, polyphosphonates such as polyvinylphosphonates, as well as copolymers, salts, derivatives, and combinations of the preceding, among various others.

As noted above, charged polymers include those containing one or more types of charged amino acids, including those which comprise a net cationic or anionic charge at neutral pH values (e.g., in the range of pH 6.5 to 7.5), including physiological pH (pH 7.4). Polyamino-acid containing-polymers which are positively charged at physiological pH generally include those containing a preponderance of one or more types of basic amino acids (e.g., lysine, arginine, ornithine, etc.). Polyamino-acid-containing polymers which are negatively charged at physiological pH generally include those containing a preponderance of one or more types of acidic amino acids (e.g., glutamic acid, aspartic acid, etc.).

As indicated noted above, coating regions in accordance with the invention comprise charged polyamino-acid-containing polymers. Such polyamino-acid-containing polymers include biodegradable and biostable polyamino acids. Polyamino-acid-containing polymers include those that contain traditional peptide-based polyamino acids. Polyamino-acid-containing polymers also include polyamino acids such as those described in U.S. Pat. No. 4,638,045 to Kohn et al., which contain amino acids that are polymerized via hydrolytically labile bonds at their respective side chains. Polyamino-acid-containing polymers further include pseudo-polyamino acids having ester bonds within the polymer backbone, which may be formed, for example, from N-protected hydroxy amino acids such as those based on tyrosine, threonine, serine and/or hydroxyproline (e.g., trans-4-hydroxy-L-proline), and which may subsequently be deprotected.

Typically, the charged polyamino-acid-containing polymers are not full length proteins, although they may vary widely in length. Commonly, lengths range from 1 kDalton or less to 1000 kDaltons or more, for example, ranging from 1 kDalton to 2.5 kDaltons to 5 kDaltons to 10 kDaltons 25 kDaltons to 50 kDaltons to 100 kDaltons to 250 kDaltons to 500 kDaltons to 1000 kDaltons.

Charged polyamino-acid-containing polymers in accordance with the present invention may also comprise polyamino acid sequences (which may be charged or uncharged) that promote cell coverage (e.g., cell binding, cell proliferation, etc.).

Specific examples of polyamino acid sequences that promote cell coverage include, for example, those containing RGD sequences (e.g., GRGDS) and WQPPRARI sequences, which have be reported to direct spreading and migrational properties of endothelial cells. See V. Gauvreau et al., Bioconjug Chem., 2005 September-October, 16 (5), 1088-97. Further examples include polyamino acid sequences containing REDV tetrapeptide, which has been shown to support endothelial cell adhesion but not that of smooth muscle cells, fibroblasts, or platelets, and YIGSR pentapeptide, which has been shown to promote epithelial cell attachment, but not platelet adhesion. Further information on REDV, YIGSR, RGD and cyclic-RGD peptides can be found in U.S. Pat. No. 6,156,572, Pub. No. US 2003/0087111, B. P. Chan, Journal of Biomedical Materials Research Part B: Applied Biomaterials, 72B (1) (2004) 52-63, Y. Xiao et al., Biophysical Journal, 71 (1996) 2869-2884 and S. P. Massia et al., The Journal of Biological Chemistry, 267 (20) (1992) 14019-14026. In addition to YIGSR, the RYVVLPR and TAGSCLRKFSTM peptide sequences have been shown to promote specific biological activities including endothelial cell adhesion. See, e.g., E. Genove et al., Biomaterials 26 (2005) 3341-3351 and the references cited therein. These sequences are present in two major protein components of the basement membrane, laminin 1 (YIGSR, RYVVLPR) and collagen IV (TAGSCLRKFSTM). Id. A further example of a cell-adhesive sequence is NGR tripeptide, which has been reported to bind to CD13 of endothelial cells. See, e.g., L. Holle et al., “In vitro targeted killing of human endothelial cells by co-incubation of human serum and NGR peptide conjugated human albumin protein bearing alpha (1-3) galactose epitopes,” Oncol. Rep. 2004 March; 11 (3):613-6. Positively charged polyamino acid sequences have been proposed for binding negatively charged sulfate and carboxylate groups of cell surface proteoglycans, examples of which include PRRARV (derived from fibronectin), PRRGRV (derived from fibronectin), YEKPGSPPREVVPRPRPGV (derived from fibronectin), RPSLAKKQRFRHRNRKGYRSQRGHSRGR (derived from vitronectin), RIQNLLKITNLRIKFVK (derived from laminin), and RYVVLPRPVCFEKGMNYTVR (derived from laminin). See, e.g., Stephen P. Massia et al., The Journal of Biological Chemistry, 267 (14), 1992, 10133-10141 and the references cited therein for further discussion of these sequences.

In some embodiments, to maximize interactions between the polyamino acid sequences that promote cell coverage and surrounding cells in the body, charged polyamino-acid-containing polymers containing such sequences may constitute the outermost layer that is deposited during layer-by-layer processing.

At physiological pH, polyamino acid sequences that promote cell coverage (e.g., those described above, among others) may have a positive net charge, a negative net charge, or a neutral net charge (e.g., uncharged or zwitterionic sequences). To the extent that it is desirable to increase or decrease the charge of polyamino-acid-containing polymers containing such sequences, one may further provide the polymers with positively charged polymer chains (e.g., polyamino acid chains containing a preponderance of basic amino acids such as those above, among others) or negatively charged polymer chains (e.g., polyamino acid chains containing a preponderance of acidic amino acids such as those above, among others).

As a specific example, one or more polyamino acid sequences that promote cell coverage (see, e.g., those described below, among others) may be provided within a polymer that also contains, for instance, a poly(aspartic acid) or poly(glutamic acid) sequence in order to render the overall net charge of the polymer more negative. Conversely, a polyamino acid sequence that promotes cell coverage may be provided within a polymer that also contains, for instance, a polylysine or polyarginine sequence in order to render the overall net charge of the polymer more positive. As noted above, such polyanionic and polycationic sequences may vary widely in length, typically ranging form 1 kDalton to 1000 kDaltons in length.

The presence of polycationic sequences may, for example, enhance binding to negatively charged sulfate and carboxylate groups of cell surface proteoglycans. Moreover, because these sequences commonly contain primary or secondary amines, they may also be employed as carriers of nitric oxide, as described in more detail below.

Peptide sequences such as those described above may be isolated from natural sources, may be formed using recombinant DNA techniques, or may be formed using synthetic techniques. As an example of the latter, peptides may be made by the “Fmoc” synthesis technique in which the carboxyl group of an N-protected amino acid is activated and reacted with the terminal primary amino group of a resin-bound amino acid/peptide, resulting in amide bond formation. Solid phase chemistry is typically used because it allows control over the peptide sequence. For further information, see, e.g., Lee Ayres, From structural proteins to synthetic polymers, Doctoral Thesis, Radboud Universiteit Nijmegen, 2005, ISBN 9090198075, Chapter 1 and the references cited therein.

As indicated above, in some embodiments, the coating regions of the present invention may comprise a charged polymer that releases nitric oxide (NO), which NO-releasing polymer may or may not be a charged polyamino-acid-containing polymer.

For example, in some embodiments, the coating regions of the present invention may comprise a charged polymer which is either an NO-releasing polymer or which is may be converted into an NO-releasing polymer, for instance by reaction with a suitable species (e.g., nitric oxide, sodium nitrite, etc.) under suitable conditions. Thus, in some embodiments, coating regions may be formed using charged NO releasing polymers. In other embodiments, coating regions may be formed using charged polymers, which are subsequently converted (within the coating) into NO releasing polymers. A few examples of NO-releasing polymers will be described in the following paragraphs.

NO-releasing species may be formed, for instance, by the reaction of secondary amine structures with two moles of NO(g) under high pressure to create a relatively stable diazeniumdiolate adduct structure. See, e.g., See M. C. Frost et al., Biomaterials 26 (2005) 1685-1693, and the references cited therein. The diazeniumdiolate adduct, which is negatively charged, requires a countercation to fulfill electroneutrality conditions, which cation can either be (a) an exogenous cation (e.g., Na⁺, NH₄⁺, etc.) or (b) an organic amine cation arising from another amine species present within the same molecule, yielding zwitterionic species. Id.

An example of a secondary amine containing amino acid is proline. Id. Thus, in certain embodiments, charged polyamino acid containing polymers for use in the invention may comprise one or more proline residues.

An example of a non-peptide polymer which can be treated with NO to form a diazeniumdiolate NO donor is polyethyleneimine (PEI). Id. PEI is a branched polymer that contains a combination of primary, secondary and tertiary amines. D. J. Smith et al., J. Med. Chem., 39 (5), 1148-1156, 1996, report that a cross-linked poly(ethylenimine), which had been exposed to NO, provides sustained NO release for 5 weeks in pH 7.4 buffer at 37° C. PEI may also be employed to form NO releasing coatings, in accordance with the present invention.

Peptides comprising amino acids with pendant primary amine groups (e.g., lysine) have also been reported to form diazeniumdiolate NO donors. For example, diazeniumdiolates may be formed by dissolving a lysine-containing peptide in deionized water and reacting it with NO at room temperature under argon gas overnight as described in Ho-Wook Jun et al., Biomacromolecules, 6 (2005) 838-844. See also, L. J. Taite et al., Journal of Biomaterials Science, Polymer Edition, 17 (10), 2006, 1159-1172, wherein poly(ethylene glycol)-lysine dendrimers were reacted with NO gas in water under argon at room temperature overnight. NO release from these materials occurred for up to 60 days under physiological conditions. As another example, J. A. Hrabie et al., “Conversion of proteins to diazeniumdiolate-based nitric oxide donors” Bioconjug. Chem. 10 (5), 1999, 838-842, describe a process for producing a reagent that is capable of transferring a nitric oxide (NO)-donating diazeniumdiolate group to lysine residues contained in proteins. Diazeniumdiolated bovine serum albumin and diazeniumdiolated human serum albumin produced by this process, upon dissolution in pH 7.4 phosphate buffer at 37° C., gradually released their NO with half-lives on the order of 3 weeks. The foregoing processes may also be employed to form NO releasing polymers from L-lysine containing peptides, in accordance with the present invention.

In other embodiments, NO-releasing peptides may be formed from peptides that comprise one or more thiol-containing amino acid such as cysteine and or homocysteine. For example, U.S. Pat. No. 5,385,937 to Stamler et al. describes the preparation of S-nitroso-homocysteine in a nitrosylation method in which homocysteine is treated with acidified sodium nitrite (NaNO₂). U.S. Pat. No. 5,593,876 to Stamler et al. describes nitrosylation of protein thiols using the same technique. See also K. S. Bohl Masters et al., J. Biomater. Sci. Polymer Edn, 16 (5), 2005, 659-672.

In certain embodiments, coating regions in accordance with the present invention may optionally include at least one therapeutic agent.

For example, in embodiments where a vascular medical device is employed, a therapeutic agent may be employed which is, for instance, an antirestenotic agent or an agent that promotes attachment and/or growth of endothelial cells. “Therapeutic agents”, “pharmaceuticals,” “drugs” and other related terms may be used interchangeably herein. Therapeutic agents may be themselves pharmaceutically active, or they may converted in vivo into pharmaceutically active substances (e.g., they may be prodrugs).

In some embodiments, the optional therapeutic agent may be a charged therapeutic agent. By “charged therapeutic agent” is meant a therapeutic agent that has an associated charge, in which case it may be introduced into the coating during the coating formation process.

A therapeutic agent may have an associated charge, for example, because it is inherently charged (e.g., because it has acidic and/or or basic groups, which may be in salt form). A few examples of inherently charged cationic therapeutic agents include amiloride, digoxin, morphine, procainamide, and quinine, among many others. Examples of anionic therapeutic agents include heparin and DNA, among many others.

A therapeutic agent may have an associated charge because it has been chemically modified to provide it with one or more charged functional groups. For instance, conjugation of water insoluble or poorly soluble drugs, including anti-tumor agents such as paclitaxel, to hydrophilic polymers has recently been carried out in order to solubilize the drug (and in some cases to improve tumor targeting and reduce drug toxicity). Similarly cationic or anionic versions of water insoluble or poorly soluble drugs have also been developed. Taking paclitaxel as a specific example, various cationic forms of this drug are known, including paclitaxel N-methylpyridinium mesylate and paclitaxel conjugated with N-2-hydroxypropyl methyl amide, as are various anionic forms of paclitaxel, including paclitaxel-poly(l-glutamic acid), paclitaxel-poly(l-glutamic acid)-PEO. See, e.g., U.S. Pat. No. 6,730,699; Duncan et al., Journal of Controlled Release 74 (2001) 135; Duncan, Nature Reviews/Drug Discovery, Vol. 2, May 2003, 347; Jaber G. Qasem et al, AAPS PharmSciTech 2003, 4 (2) Article 21. In addition to these, U.S. Pat. No. 6,730,699, also describes paclitaxel conjugated to various other charged polymers (e.g., polyelectrolytes) including poly(d-glutamic acid), poly(dl-glutamic acid), poly(l-aspartic acid), poly(d-aspartic acid), poly(dl-aspartic acid), poly(l-lysine), poly(d-lysine), poly(dl-lysine), copolymers of the above listed polyamino acids with polyethylene glycol (e.g., paclitaxel-poly(l-glutamic acid)-PEO), as well as poly(2-hydroxyethyl 1-glutamine), chitosan, carboxymethyl dextran, hyaluronic acid, human serum albumin and alginic acid. Still other forms of paclitaxel include carboxylated forms such as 1′-malyl paclitaxel sodium salt (see, e.g. E. W. DAmen et al., “Paclitaxel esters of malic acid as prodrugs with improved water solubility,” Bioorg Med. Chem., 2000 February, 8 (2), pp. 427-32). Polyglutamate paclitaxel, in which paclitaxel is linked through the hydroxyl at the 2′ position to the A carboxylic acid of the poly-L-glutamic acid (PGA), is produced by Cell Therapeutics, Inc., Seattle, Wash., USA. (The 7 position hydroxyl is also available for esterification). This molecule is said to be cleaved in vivo by cathepsin B to liberate diglutamyl paclitaxel. In this molecule, the paclitaxel is bound to some of the carboxyl groups along the backbone of the polymer, leading to multiple paclitaxel units per molecule. For further information, see, e.g., R. Duncan et al., “Polymer-drug conjugates, PDEPT and PELT: basic principles for design and transfer from the laboratory to clinic,” Journal of Controlled Release 74 (2001) 135-146, C. Li, “Poly(L-glutamic acid)-anticancer drug conjugates,” Advanced Drug Delivery Reviews 54 (2002) 695-713; Duncan, Nature Reviews/Drug Discovery, Vol. 2, May 2003, 347; Qasem et al, AAPS PharmSciTech 2003, 4 (2) Article 21; and U.S. Pat. No. 5,614,549. Such strategies may be applied to a host of other therapeutic agents, including anti-restenotic agents other than paclitaxel, for instance, olimus family drugs such as everolimus.

Using the above and other strategies, paclitaxel and many other therapeutic agents may be covalently linked or otherwise associated with a variety of charged species, including charged polymers, thereby forming charged drugs and prodrugs.

A therapeutic agent may also have an associated charge because it is attached to a charged particle or because it is encapsulated within a charged particle, for example, encapsulated within a charged nanocapsule or within a charged micelle, among others. A therapeutic agent may be provided within a charged capsule, for example, using layer-by-layer techniques in which capsules are formed from alternating layers of polyanions and polycations such as those described above and in Pub. No. US 2005/0129727 to Weber et al. For a specific example of such a technique, see I. L. Radtchenko et al., “A novel method for encapsulation of poorly water-soluble drugs: precipitation in polyelectrolyte multilayer shells,” International Journal of Pharmaceutics, 242 (2002) 219-223.

Using the above and other techniques, a wide range of therapeutic agents may be provided with associated charges.

As previously indicated, coating regions in accordance with the invention may be formed by repeated exposure to alternating, oppositely charged species in what is known as layer-by-layer deposition. The layers self-assemble by means of electrostatic interactions, thus forming a coating region over the substrate. In a typical layer-by-layer deposition technique, coating growth proceeds through sequential steps, in which the substrate is exposed to solutions or suspensions of cationic or anionic species, frequently with intermittent rinsing between steps.

Layer-by-layer assembly may be conducted, for example, by sequentially exposing a selected substrate to solutions or suspensions that contain species of alternating net charge, including solutions or suspensions that contain one or more of the following charged species, among others: (a) charged polyamino-acid-containing polymers (e.g., polymers that contain peptides that promote cell attachment and/or proliferation, etc.), (b) charged polymers that release NO or which can be converted into NO releasing polymers after deposition (e.g., a polyamine that can be exposed to NO or another diazeniumdiolate forming species, a polythiol that can be exposed to sodium nitrite to form an S-nitrosothiol, etc.), (c) charged polymers, which are not polyamino-acid-containing polymers and which are not NO releasing polymers or their precursors (e.g., selected from those polyelectrolytes described above, among others), and (d) charged therapeutic agents.

The concentration of the charged species within these solutions and suspensions can vary widely, with typical values being on the order of from 0.01 to 10 mg/ml, among others.

Moreover the pH of these solutions and suspensions may be set as desired. Buffer systems may be employed for this purpose, if desired. The charged entities chosen may be ionized at neutral pH (e.g., at pH 6.5-7.5) or at the pH of the body location where the device is to be inserted or implanted (e.g., physiological pH), among other possibilities.

The solutions and suspensions containing the charged species may be applied to the substrate surface using a variety of techniques including, for example, full immersion techniques such as dipping techniques, spraying techniques, roll and brush coating techniques, techniques involving coating via mechanical suspension such as air suspension, ink jet techniques, spin coating techniques, web coating techniques, polymer stamping, and combinations of these processes. The choice of the technique will depend on the requirements at hand. For example, full immersion techniques may be employed where it is desired to apply the species to an entire substrate, including surfaces that are hidden from view (e.g., surfaces which cannot be reached by line-of-sight techniques, such as spray techniques). On the other hand, techniques such as spraying, roll coating, brush coating, ink jet printing, and stamping may be employed, for instance, where it is desired to apply the species only certain portions of the substrate. As a specific example, medical devices (e.g., tubular implants, such as stents and grafts) may be produced in which only the solid-tissue-contacting areas (e.g., the outer surface of the stent or the inner surface of the graft) are provided with a therapeutic agent, for example, an antirestenotic agent.

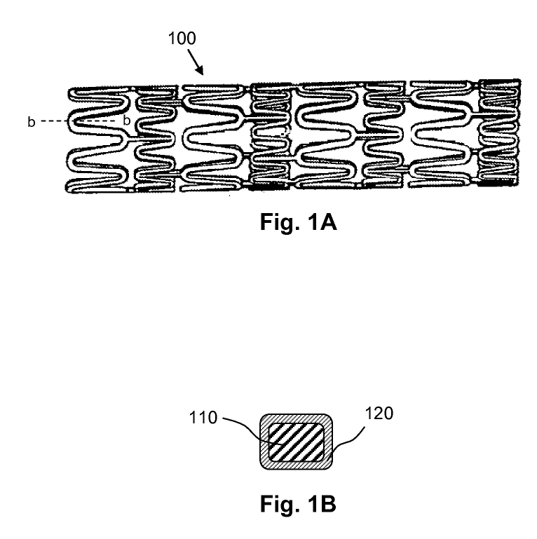

A specific embodiment of the invention will now be described with reference to the Figures. Referring now to FIGS. 1A and 1B, a stent 100 is shown, in accordance with an embodiment of the present invention. As seen from FIG. 1B, which is a cross section taken along line b-b of FIG. 1A, the stent 100 comprises a substrate 110, which may be, for example, a biostable metallic substrate such as a nitinol or stainless steel substrate or a bioresorbable metallic substrate such as iron, magnesium, zinc or their alloys, among others. Disposed over the substrate is a coating region 120 in accordance with the present invention. The coating region 120 may be formed, for example, by first dipping the substrate in a solution of a readily adsorbable polyelectrolyte such as PEI or PAH, followed by alternatively dipping the substrate in a first solution containing an anionic charged polymer selected, for example, from l-glutamic acid polymers, including those that further contain amino acid sequences that promote cell coverage (e.g., RGD, etc.), heparin, hyaluronic acid, alginic acid, dextran sulfate, cellulose sulfate, and poly(styrene sulfonate), and a second solution containing an cationic charged polymer selected, for example, from l-lysine polymers, including those that further contain amino acid sequences that promote cell coverage, chitosan, protamine sulfate, polyvinyl pyridine, poly(allylamine hydrochloride), and polydiallydimethylammonium chloride (PDADMAC). For instance, the anionic charged polymer may be an anionic polyamino-acid-containing polymer that includes 50% or more l-glutamic acid moieties along with a number of RGD peptide motifs and the anionic charged polymer may be poly-l-lysine. Prior to implanting the stent, diazeniumdiolate NO donors may be formed, for example, by reacting the poly-l-lysine with NO, for example, as described in Ho-Wook Jun et al., supra.

Although various embodiments of the invention are specifically illustrated and described herein, it will be appreciated that modifications and variations of the present invention are covered by the above teachings without departing from the spirit and intended scope of the invention.

1. An implantable or insertable medical device comprising: (a) a substrate and (b) a coating region that comprises (i) a charged poly(amino acid) containing polymer having a first net charge and (ii) an additional charged polymer of opposite net charge, wherein the charged poly(amino acid) containing polymer comprises an integrin binding sequence, wherein the charged poly(amino acid) containing polymer releases NO upon implantation or insertion in vivo, or both.

2. The medical device of claim 1, wherein the medical device is a vascular stent.

3. The medical device of claim 1, wherein the coating region comprises at least 5 layers that comprise said charged poly(amino acid) containing polymer in alternation with at least 5 layers that comprise said additional charged polymer.

4. The medical device of claim 1, wherein the coating region is bioresorbable.

5. The medical device of claim 1, wherein the charged poly(amino acid) containing polymer is between 1 kDalton and 1000 kDaltons in molecular weight.

6. The medical device of claim 1, wherein the charged poly(amino acid) containing polymer comprises a peptide sequence that promotes cell coverage.

7. The medical device of claim 6, wherein the peptide sequence is selected from

| | |
|---|---|
| RGD, REDV, | (SEQ ID NO: 1) |
| YIGSR, | (SEQ ID NO: 2) |
| RYVVLPR, | (SEQ ID NO: 3) |
| TAGSCLRKFSTM, | (SEQ ID NO: 4) |
| WQPPRARI, | (SEQ ID NO: 5) |
| PRRARV, | (SEO ID NO: 6) |
| PRRGRV, | (SEO ID NO: 7) |
| YEKPGSPPREVVPRPRPGV, | (SEQ ID NO: 8) |
| RPSLAKKQRFRHRNRKGYRSQRGHSRGR, | (SEQ ID NO: 9) |
| RIQNLLKITNLRIKFVK | (SEQ ID NO: 10) |
| and | |
| RYVVLPRPVCFEKGMNYTVR. | (SEQ ID NO: 11) |

8. The medical device of claim 6, wherein the additional charged polymer is an NO releasing polymer.

9. The medical device of claim 8, wherein the additional charged polymer comprises a sequence selected from a polyethyleneimine sequence, a polyproline sequence, a polylysine sequence, a polyarginine sequence, and a polycysteine sequence.

10. The medical device of claim 1, wherein the charged poly(amino acid) containing polymer releases NO upon implantation or insertion in vivo.

11. The medical device of claim 10, wherein the charged poly(amino acid) containing polymer comprises a sequence selected from a polyproline sequence, a polylysine sequence, a polyarginine sequence, a poly(lysine-co-arginine) sequence, and a polycysteine sequence.

12. The medical device of claim 1, wherein the additional charged polymer is an NO releasing polymer.

13. The medical device of claim 1, wherein the charged poly(amino acid) containing polymer has a positive net charge and the additional charged polymer has a negative net charge.

14. The medical device of claim 13, wherein the charged poly(amino acid) containing polymer comprises an amino acid sequence selected from a polylysine sequence, a polyarginine sequence and a poly(lysine-co-arginine) sequence.

15. The medical device of claim 13, wherein the additional charged polymer comprises a sequence selected from a hyaluronic acid sequence, a polyaspartic acid sequence, a polyglutamic acid sequence and a poly(aspartic acid-co-glutamic acid) sequence.

16. The medical device of claim 1, wherein the charged poly(amino acid) containing polymer has a negative net charge and the additional charged polymer has a positive net charge.

17. The medical device of claim 16, wherein the charged poly(amino acid) containing polymer comprises a sequence selected from a polyaspartic acid sequence, a polyglutamic acid sequence and a poly(aspartic acid-co-glutamic acid) sequence.

18. The medical device of claim 16, wherein the charged additional polymer comprises a sequence selected from a polyethyleneimine sequence, a polylysine sequence, a polyarginine sequence and a poly(lysine-co-arginine) sequence.

19. The medical device of claim 1, wherein the charged poly(amino acid) containing polymer comprises a peptide sequence that promotes cell coverage and releases NO upon implantation or insertion in vivo.

20. The medical device of claim 1, wherein the coating region is formed by alternating exposure to a first solution comprising the charged poly(amino acid) containing polymer and a second solution comprising the additional charged polymer.
